(19) United States
(12) Patent Application Publication
(10) Pub. No.: US 2015/0080747 A1
(43) Pub. Date: Mar. 19, 2015

(54) DEVICES, SYSTEMS AND METHODS FOR LOCALLY MEASURING BIOLOGICAL CONDUIT AND/OR LESION COMPLIANCE, OPPOSITION FORCE AND INNER DIAMETER OF A BIOLOGICAL CONDUIT

(71) Applicant: Cardiovascular Systems, Inc., St. Paul, MN (US)

(72) Inventor: Victor Leo Schoenle, Greenfield, MN (US)

(21) Appl. No.: 14/315,774

(22) Filed: Jun. 26, 2014

Related U.S. Application Data
(60) Provisional application No. 61/840,693, filed on Jun. 28, 2013.
(60) Provisional application No. 61/871,529, filed on Aug. 29, 2013.

(51) Int. Cl. A61B 5/02; A61M 25/10; A61B 5/00

(52) U.S. Cl. CPC A61B 5/02007; A61B 5/6853; A61M 25/10182
USPC 600/481

(57) ABSTRACT

The present invention is directed in various methods, devices and systems relating to systems and methods for measuring and calculating a biological conduit's, e.g., a blood vessel, inner diameter, the opposition force of the conduit's wall on an expanding balloon disposed therein and the conduit and/or lesion compliance to the expanding balloon.

### CROSS-REFERENCE TO RELATED APPLICATIONS

This application claims priority to App. Ser. No. 61/840,693, entitled “Inflation Device That Automatically Controls and Measures Applied Force With Wireless Control of Functions,” filed Jun. 28, 2013, and to App. Ser. No. 61/871,529, entitled “Devices, Systems and Methods for a Device and Method for Quantifying Lumen Internal Diameter and Vessel Compliance,” filed Aug. 29, 2013, the entire contents of each of which are hereby incorporated by reference.

### FIELD OF THE INVENTION

The present disclosure generally relates to methods, devices and systems relating to removing occlusions from vessels. More specifically, the present invention comprises measuring an opposition force encountered by an inflating balloon within a biological conduit, e.g., a blood vessel, and quantifies the biological conduit's inner diameter and compliance and/or compliance of a lesion within the conduit, at the location where the opposition force is measured.

### DESCRIPTION OF THE RELATED ART

A variety of techniques and instruments have been developed for use in the removal or repair of tissue in arteries and similar body passageways, e.g., biological conduits. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Characterization of the compliance of the subject biological conduit, e.g., blood vessel, as well as the compliance of a lesion within the conduit, e.g., blood vessel, is a critical element. Known inflatable devices having pressure sensors incorporated thereon, with manual measurement and control of the pressure levels and inflation rate. In some cases, a syringe and associated pressure gauge is used to inflate and/or deflate the inflatable device. In the known solutions, a balloon inflation device is a hand-held device comprising a screw-driven syringe with a pressure gauge that indicates the inflation pressure than the balloon is under during operation. The operator may manually rotate the screw to the desired inflation pressure. The operator must then visually estimate how well the device is contacting the wall of the vessel and to match the device to the vessel, e.g., artery. Each time the operator requires a visualization of the vessel to device conformation, the patient must be injected with a contrast fluid with subsequent production of an x-ray film to enable the visualization. The visualization process is undesirable as it is time consuming and requires harmful drugs and x-rays. In addition, the known processes do not allow quantification of the opposition force the inflating balloon places on the wall of the vessel.

However, none of these references disclose, inter alia, a device, system or method capable of measuring an opposition force and that is further capable of quantifying the conduit's internal diameter and compliance, or the compliance of a lesion within the conduit, e.g., blood vessel, at a specific location, e.g., the site of an occlusion. This procedure may be performed, and data collected, prior to, during and/or after a procedure such as angioplasty, atherectomy and the like.

### BRIEF SUMMARY OF THE INVENTION

The present invention is directed in various methods, devices and systems relating to systems and methods for measuring and calculating a biological conduit's, e.g., a blood vessel, inner diameter, the opposition force of the conduit's wall on an expanding balloon therein and the conduit's compliance to the expanding balloon as well as the compliance of a lesion within the conduit.

### BRIEF DESCRIPTION OF THE DRAWINGS

FIG. 1 illustrates a reference inflation compliance curve for an unrestrained balloon with a fixed inflation volume and fixed inflation rate (unrestrained reference);

FIG. 2 illustrates the differences between the reference compliance curve of FIG. 1 and an inflation compliance curve from the same balloon with same fixed inflation volume and inflation rate under restrained conditions within a healthy biological conduit, e.g., a blood vessel without a lesion, (restrained reference);

FIG. 3 illustrates the inflation compliance curve of an occluded vessel pre-treatment (restrained) biological conduit, e.g., a blood vessel with a lesion, compared with the unrestrained balloon reference curve from FIG. 1 and the restricted healthy biological conduit reference curve data from FIG. 2 with the same fixed inflation volume and rate;

FIG. 4 illustrates the inflation compliance curve of an occluded biological conduit, e.g., a blood vessel with a lesion, post-treatment (restrained) compared with the unrestrained balloon reference curve from FIG. 1 and the restricted healthy vessel reference curve data from FIG. 2 with the same fixed inflation volume and rate; and

FIG. 5 illustrates one embodiment of a device and system of the present invention.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

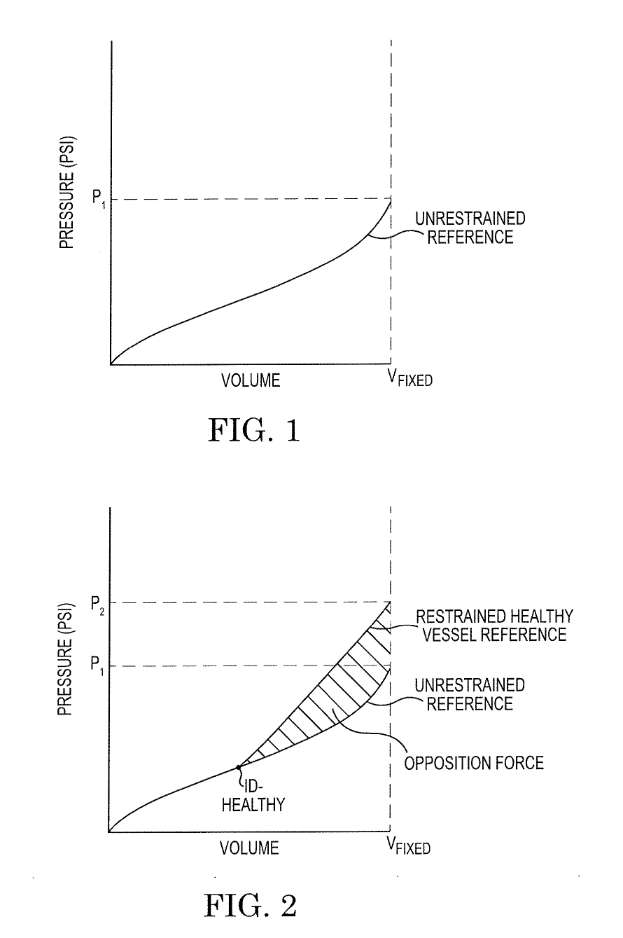

FIG. 1 illustrates the development of an unrestrained reference compliance curve using an unrestrained balloon and a fixed inflation volume with a fixed inflation rate. Thus, the pressure measured by a transducer that is operationally attached to the balloon and as will be discussed later is recorded and graphed on the y-axis, while the volume added to the balloon during the inflation process is recorded and graphed on the x-axis. The total volume is fixed (V-Fixed) as is the inflation rate. This process is completed without any restrictive forces on the balloon such as a vessel wall during the inflation process.

The result is a reference compliance inflation curve for a particular balloon, or a balloon having a particular set of characteristics, e.g., size, shape, elasticity. Because the balloon is unrestrained and both the volume and the inflation rate are fixed, it is possible to measure, and record, the outer diameter (OD) of the balloon throughout the inflation process, i.e., the OD of the unrestrained balloon at any point in the inflation process can be mapped to a particular set of pressure, volume coordinate data. The OD data is recorded along with the pressure and volume data for future reference. The OD data may be used to quantify the internal diameter of any biological conduit, e.g., a blood vessel, that the balloon is expanded within as further described below.

FIG. 2 illustrates the development of a restrained healthy biological conduit, e.g., blood vessel, reference compliance inflation curve using a balloon with the same physical characteristics as that used to develop the unrestrained balloon compliance curve of FIG. 1 as well as the same fixed volume and inflation rate used for the unrestrained reference compliance curve of FIG. 1. The remaining disclosure refers to the subset of blood vessels within the broader category biological conduit which is broadly defined herein as a channel with boundaries or walls within a mammal. This reference is solely for ease of disclosure and not intended to limit the disclosure to blood vessels in any way. The restrained healthy vessel reference compliance curve information relating to the pressure measured by the operationally attached pressure transducer is captured, recorded and graphed against the fixed volume that is infused into the restrained balloon at a fixed inflation rate.

FIG. 2 also comprises unrestrained reference compliance curve data for the same balloon, or one with the same physical characteristics, and for the same fixed volume and inflation rate as used for the restrained reference compliance curve data generation.

Several significant features appear on FIG. 2. First, as the fixed volume is reached, it is clear that the pressure measured within the unrestrained reference at P1, is lower than the pressure measured within the restrained reference at P2. This is the effect of restraint on the inflation. Similarly, the volume changes at a given pressure may also be monitored.

Additionally, following the data from the origin, a point of divergence is reached, where the restrained reference begins to experience higher pressure than the unrestrained reference. This point of divergence is marked on FIG. 2 as ID-Healthy and represents the expansion point at which the restrained balloon encounters resistance in the form of the healthy vessel wall it is expanding within. Stated differently, the expanding balloon first experiences an opposition force at ID-Healthy as a consequence of the expanding balloon encountering the inner diameter of the healthy vessel wall. Consequently, it is now possible to determine the internal diameter of the vessel at the location of the expanding balloon, by comparing the compliance curve of FIG. 2 with the unrestrained reference compliance curve of FIG. 1 and locating the point of divergence marked as ID-Healthy. Next, reference may be made to the previously mapped set of OD's corresponding to a given volume and pressure along the unrestrained reference compliance curve of FIG. 1 and as described above to determine the outer diameter of the restrained healthy vessel reference balloon at ID-Healthy. The outer diameter of the restrained healthy vessel reference balloon at ID-Healthy is the same as the inner diameter of the healthy vessel wall.

Further, the present invention is enabled to measure a quantity defined herein as opposition force, i.e., the force applied by the vessel wall against the expanding balloon, a force not experienced by the unrestrained reference balloon of FIG. 1. This is illustrated graphically by the shaded area in FIG. 2 between the restrained reference compliance curve and the unrestrained reference compliance curve after the point of divergence ID-Healthy discussed above. The opposition “force” quantity may be calculated as a surrogate to force through use of the pressure values. For example, in FIG. 2, at V-Fixed, the opposition force may be characterized as delta P or P2−P1. This calculation may be made at any point in the inflation process for any given volume. Alternatively, the pressures at any given volume within the inflation process may be converted to actual force by dividing the pressure for the restrained and unrestrained reference compliance curves at any point beyond the point of divergence by the surface area of the inflating balloon, a known and/or measurable quantity, and computing the difference between restrained reference force and unrestrained reference force. Still more alternatively, the area between the restrained reference compliance curve and the unrestrained reference compliance curve beyond the point of divergence may be calculated using known mathematical techniques in order to calculate the total opposition force.

Moreover, it is possible to measure the elasticity, or compliance, of the restrained reference compliance curve vessel, based on the slope of the restrained reference compliance curve, i.e., the change in pressure compared with the change in volume, as compared with the slope of the unrestrained reference compliance curve, beginning at the point where the pressure within that restrained reference vessel reaches the point of divergence ID-Healthy discussed above. The steeper the slope of the restrained reference compliance curve as compared with the unrestrained reference compliance curve, the less elastic or compliant is the restraining vessel that the restrained reference balloon is expanding within. In contrast, a slope that is less steep for the restrained compliance curve as compared with the unrestrained reference compliance curve indicates a more compliant, or elastic, vessel. Note that in this case, the restrained reference vessel is healthy and, therefore, the compliance measurement is only for the vessel and not a lesion therein. Compliance, or elasticity, may be measured and/or quantified by comparing the volume changes at given pressures. Alternatively, compliance or elasticity may be quantified by comparing the pressure changes at given volumes. Either of these methods may be evaluated using a slope comparison.

Note further that the restrained healthy vessel reference compliance curve may be generated within a patient in the same vessel that is occluded, but in a relatively healthy section. Alternatively, another similar vessel within the patient may be used to generate the reference data. Still more alternatively, laboratory measurements may be conducted using sleeves of known elasticity in order to build a reference library of incremental volumes, infusion rates and matching those variables in a test matrix against sleeves of incremental elasticity. Herein, elasticity is defined as compliance and the two terms may be used interchangeably. Generally, elasticity, or compliance, is the ability of the vessel, or sleeve, to accommodate, i.e., increase in inner diameter, with an increasing volume and resulting increase in pressure. All of these reference library data may be stored in a database that is accessible for comparison purposes during an actual working procedure such as an atherectomy procedure, stent delivery or transcatheter aortic valve replacement (TAVR), and the like to enable the operator to determine real-time progress and sufficiency of the procedure for inner diameter changes, opposition force changes and/or compliance, i.e., elasticity, of the subject biological conduit. In short, the present invention may be used alone or in combination with any procedure that desires data on a conduit's inner diameter and changes thereof, opposition force changes and compliance of the conduit and/or lesion when present.

It is known that a healthy artery, e.g., has an approximate 5 to 7% compliance, or elasticity, when subjected to approximately 100 mg of pressure. This is generally the range required by a healthy artery to accommodate pressure and volume changes at the extremes of physical exertion, i.e., from sleeping to rigorous exercise. Thus, vessels with healthy compliance will experience changes in the inner diameter during increases in pressure and/or volume. Consequently, increases in volume are mitigated in terms of increasing pressure as the flow volume is also increased due to the larger channel. In contrast, vessels lacking healthy compliance will resist changes in inner diameter accommodation in response to increases in pressure and/or volume. Consequently, unhealthy vessels may retain a static diameter during changes in volume which drives pressures to potentially unhealthy levels.

Vessels having occlusions may exhibit these non-compliant properties, in addition to having inner diameters that are smaller than normal due to the occlusive material. Procedures to remove the occlusion, e.g., rotational and/or orbital atherectomy, may be employed to increase the inner diameter of the vessel at the previously partially or completely occluded location as well as to remove the material bound to the inner wall of the vessel which may contribute to a loss of compliance or elasticity.

Further, in some cases, an unrestrained reference compliance curve(s) may be used for analytical comparison against test data without additional use of a restrained healthy vessel reference compliance curve(s). In other cases, a restrained reference compliance curve(s) may be used for analytical comparison against test data without additional use of an unrestrained compliance curve(s). In still other cases, both an unrestrained reference compliance curve and a restrained healthy vessel reference compliance curve may be used to compare against test data. The reference compliance curve data, whether restrained or unrestrained, may be tabulated and stored in a database and/or in the memory of an external device such as a programmable computer or similar device. This data may thus be accessed for comparative purposes as will be discussed herein. In all cases, the present invention may be used to quantify compliance of the biological conduit, e.g., a blood vessel, and/or a lesion that is within the conduit.

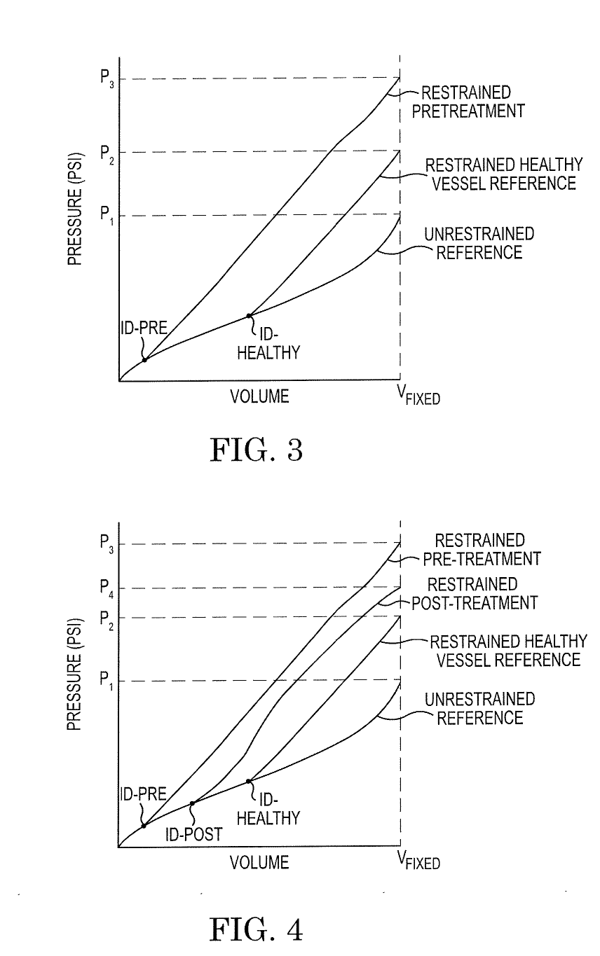

Turning now to FIG. 3, a compliance inflation curve for a test occluded vessel is illustrated as restrained (pre-treatment) in combination with the restrained and unrestrained reference compliance curves discussed above. Pre-treatment indicates that, e.g., an occlusion is present and the removal process or treatment has not occurred. The balloon, matching that of one or both of the reference compliance curves (when both the unrestrained and restrained compliance curves are used) is employed together with the same fixed volume and inflation rate parameters used to generate the reference compliance curve(s) used. The unrestrained reference compliance curve and/or the restrained reference compliance curve may be used as illustrated. In some embodiments, as discussed above, the reference compliance curve(s) may be pre-stored in a database and/or memory of a computing device and accessible during the generation of the test data as in FIG. 3 for comparative analysis.

Analysis of the restrained pre-treatment vessel data proceeds in a similar fashion as discussed above when comparing the restrained and unrestrained reference compliance curves. The point of divergence of pressures at a given volume for the test restrained pre-treatment vessel occurs at a smaller volume than either the restrained healthy vessel reference or the unrestrained reference compliance curves. This point of divergence is marked as ID-pre and indicates the inner diameter for the restrained pre-treatment vessel, as derived from the restrained healthy vessel reference compliance curve and the unrestrained reference compliance curve. ID-pre is graphically smaller than ID-healthy. The data also indicates the relative size of the inner diameter of the restrained healthy reference compliance curve, marked as ID-healthy as indicated by its divergence of pressure at a given volume compared with the unrestrained reference compliance curve. Thus, a comparison may now be made between the healthy vessel inner diameter and the restrained pre-treatment vessel inner diameter which is clearly smaller than the healthy vessel's inner diameter as shown graphically in FIG. 3. The method for determining the inner diameter of the test vessel is done with comparison and reference to the OD table developed for any given volume and pressure for the unrestrained reference compliance curve as discussed above. Since the test and unrestrained reference balloons are of the same physical characteristics, and filled at the same inflation rate with the same fixed volume, the outer diameters of the two balloons will be the same so long as the point of divergence ID-pre has not been reached on the graph. This indicates that the vessel wall has not been encountered and so is applying no opposition force to the expanding test balloon. The inner diameter of the wall is, as discussed above, determined from the point of divergence ID-pre, where the wall is encountered by the expanding balloon. The easy and real-time graphical visualization of the relative pressures at given volumes and the relative inner diameters for the test vessel and the healthy reference vessel is important to enable surgical operator to see how different the test site is in terms of inner diameter than compared with a similar healthy vessel. In addition, the operator may readily see the area between the test and reference curves and the relative slopes for the curves and visually ascertain compliance or elasticity as well as the opposition force metrics. Alternatively, executable instructions for calculating each of the afore-mentioned metrics may be stored in the memory of a programmable computing device and executable by a processor that is in communication with the memory for display on a display device.

Thus, a comparison of the relative measured pressures at any point beyond the point of divergence ID-pre of the restrained pre-treatment vessel pressure from the restrained healthy vessel compliance curve of FIG. 3 may also be made. Clearly the restrained pre-treatment vessel pressure P3 is higher at any given volume beyond the divergence point than either the restrained healthy reference compliance curve's pressure P2 or the unrestrained compliance curve's pressure P1.

Further, the opposition force of the balloon used to generate the compliance curve for the restrained pre-treatment vessel may now be quantified as the area between the restrained pre-treatment compliance curve and the restrained reference compliance curve, beyond the point of divergence ID-Healthy of those compliance curves. Alternatively, the opposition force may be the delta P at any given volume between the restrained test compliance curve and the restrained healthy vessel reference curve, at any point beyond ID-Healthy.

Moreover, the elasticity, or compliance, of the vessel and/or the lesion therein that comprises the occlusion and used to generate the restrained pre-treatment compliance curve of FIG. 3 may be measured by comparing the slope of that curve with the slope of the restrained healthy reference compliance curve. As one would expect, the pre-treatment vessel and/or lesion has a higher slope of pressure change with increasing volume than does the restrained healthy reference vessel. This indicates a degree of loss of elasticity or compliance in the pre-treatment vessel as a result of the presence of the lesion as compared with the reference vessel and may be calculated at any point along the compliance curves for a given volume.

FIG. 4 is similar to FIG. 3 except that now the test compliance curve is from a vessel that has some, or all, of the occlusive material removed, or undergone another procedure to increase inner diameter and/or compliance, i.e., is “post-treatment”. Thus, the pressures of the retrained (post-treatment) compliance curve, restrained (pre-treatment), restrained healthy reference compliance curve and the unrestrained compliance curve may be compared as each compliance curve is generated using the same balloon or one with similar physical characteristics, the same fixed volume and the same inflation rate.

Consequently, the restrained post-treatment compliance curve's pressure P4 is illustrated as slightly higher than the compliance curve pressure P2 generated within the restrained reference healthy vessel and higher still than the pressure P1 generated by the unrestrained reference compliance curve, at any given volume beyond the relevant point of divergence at ID-post. The compliance curve for restrained pre-treatment from FIG. 3 is included for use in comparing its pressure P3 at a given volume after the point of divergence at ID-Healthy.

In addition to relative pressure data, the present invention also allows quantitation of the inner diameters (by the relevant points of divergence) of the restrained pre-treatment (ID-pre), the restrained post-treatment (ID-post) and the healthy reference vessel (ID-Healthy). As perhaps expected, ID-healthy is slightly larger than the restrained post-treatment inner diameter, while both ID-post and ID-healthy are significantly larger than ID-pre, indicating a successful procedure is at least underway.

The test data may be captured real-time during an occlusion removal procedure, or other procedure designed to increase a vessel's diameter and/or its compliance in order to enable the graphical comparison and display as discussed above. In the case of the data of FIG. 4, the operator may determine that further atherectomy, angioplasty, or other procedure may be needed since the real-time data indicates that ID-healthy is still larger than ID-post, the opposition force for the restrained post-treatment compliance curve is larger than healthy vessel reference compliance curve. Further, the compliance or elasticity of the restrained post-treatment compliance curve, as determined by the relative steepness of its slope, may be less than the restrained healthy vessel compliance curve, thereby providing data on the compliance of the vessel and/or lesion post-treatment.

As discussed above, graphical display of the compliance curves as well as, in alternative embodiments, the calculation and display of the inner diameter, opposition force and compliance/elasticity metrics is a great aid to the operator in determining what, if any, additional work is required to optimize the occlusion removal or other similar procedure.

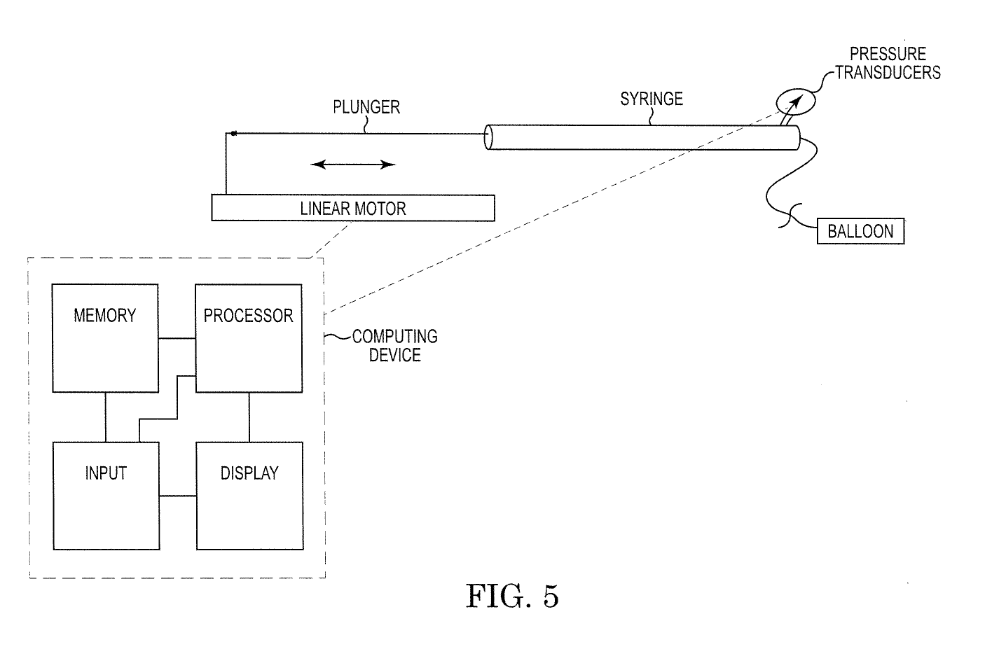

The functionality of the above method may be achieved using a variety of devices. The required elements consist of a balloon of known elasticity, or compliance, a device, e.g., a syringe, that is capable of injecting a known and fixed volume of fluid to inflate the balloon at a known and fixed rate, a pressure transducer in operative communication and connection with the inflating balloon to measure the pressure experienced by the balloon as it inflates. One such exemplary system is illustrated in FIG. 5. There is illustrated an exemplary linear motor that is capable of translating the plunger of syringe at a fixed rate. Alternative means of providing a constant, known inflation rate are also known and within the scope of the present invention. The syringe is filled with a known and fixed volume of fluid for inflating a balloon. A pressure transducer is in operative communication and connection with the balloon to measure and display and/or record the pressure data as well as the corresponding volume data.

In certain devices, a wireless control device as is known in the art may be used to control the linear motor, or other means of providing constant and known inflation rates.

The operator may also input data into the computing device, e.g., a preselected desired opposition force may be selected and input into the computing device. The result is an automatic inflation of the balloon to the selected opposition.

The device may further have the ability to learn, and store, compliance curve profiles for various balloons and device for ease of access during subsequent procedures.

Alternative devices and/or systems may be employed. For example, the pressure and volume data may be output to a programmable computing device and stored in a memory within the computing device. The stored data may be then subjected to programmable instructions that are stored within the device's memory and that, when executed by a processor in operative communication with the memory, an input such as a keyboard or the like and a graphic display, transform the data into the graphical form as illustrated in the Figures herein. The reference compliance curve(s) may also be stored in the device's memory and graphically displayed along with the test data for visual comparison with the key metrics marked and highlighted for ease of visualization. For example, the inner diameter size quantitation for the test data's compliance curve may be illustrated, pre-treatment and/or post-treatment, and compared with that of a healthy reference compliance curve, to assist in determining if the procedure is complete. Additionally, the opposition force, as describe herein, may be measured, quantified and displayed in real time to allow the operator to determine procedural progress. Moreover, the compliance, or elasticity of the vessel may be measured, quantified and graphically displayed as a slope comparison with the reference compliance curve as described herein.

Various embodiments of the present invention may be incorporated into a rotational atherectomy system as described generally in U.S. Pat. No. 6,494,890, entitled “ECCENTRIC ROTATIONAL ATHERECTOMY DEVICE,” which is incorporated herein by reference. Additionally, the disclosure of the following co-owned patents or patent applications are herein incorporated by reference in their entireties: U.S. Pat. No. 6,295,712, entitled “ROTATIONAL ATHERECTOMY DEVICE”; U.S. Pat. No. 6,132,444, entitled “ECCENTRIC DRIVE SHAFT FOR ATHERECTOMY DEVICE AND METHOD FOR MANUFACTURE”; U.S. Pat. No. 6,638,288, entitled “ECCENTRIC DRIVE SHAFT FOR ATHERECTOMY DEVICE AND METHOD FOR MANUFACTURE”; U.S. Pat. No. 5,314,438, entitled “ABRASIVE DRIVE SHAFT DEVICE FOR ROTATIONAL ATHERECTOMY”; U.S. Pat. No. 6,217,595, entitled “ROTATIONAL ATHERECTOMY DEVICE”; U.S. Pat. No. 5,554,163, entitled “ATHERECTOMY DEVICE”; U.S. Pat. No. 7,507,245, entitled “ROTATIONAL ANGIOPLASTY DEVICE WITH ABRASIVE CROWN”; U.S. Pat. No. 6,129,734, entitled “ROTATIONAL ATHERECTOMY DEVICE WITH RADIALLY EXPANDABLE PRIME MOVER COUPLING”; U.S. Pat. No. 8,597,313, entitled “ECCENTRIC ABRADING HEAD FOR HIGH-SPEED ROTATIONAL ATHERECTOMY DEVICES”; U.S. Pat. No. 8,439,937, entitled “SYSTEM, APPARATUS AND METHOD FOR OPENING AN OCCLUDED LESION”; U.S. Pat. Pub. No. 2009/0299392, entitled “ECCENTRIC ABRADING ELEMENT FOR HIGH-SPEED ROTATIONAL ATHERECTOMY DEVICES”; U.S. Pat. Pub. No. 2010/0198239, entitled “MULTI-MATERIAL ABRADING HEAD FOR ATHERECTOMY DEVICES HAVING LATERALLY DISPLACED CENTER OF MASS”; U.S. Pat. Pub. No. 2010/0036402, entitled “ROTATIONAL ATHERECTOMY DEVICE WITH PRE-CURVED DRIVE SHAFT”; U.S. Pat. Pub. No. 2009/0299391, entitled “ECCENTRIC ABRADING AND CUTTING HEAD FOR HIGH-SPEED ROTATIONAL ATHERECTOMY DEVICES”; U.S. Pat. Pub. No. 2010/0100110, entitled “ECCENTRIC ABRADING AND CUTTING HEAD FOR HIGH-SPEED ROTATIONAL ATHERECTOMY DEVICES”; U.S. Design Pat. No. D610258, entitled “ROTATIONAL ATHERECTOMY ABRASIVE CROWN”; U.S. Design Pat. No. D6107102, entitled “ROTATIONAL ATHERECTOMY ABRASIVE CROWN”; U.S. Pat. Pub. No. 2009/0306689, entitled “BIDIRECTIONAL EXPANDABLE HEAD FOR ROTATIONAL ATHERECTOMY DEVICE”; U.S. Pat. Pub. No. 2010/0211088, entitled “ROTATIONAL ATHERECTOMY SEGMENTED ABRADING HEAD AND METHOD TO IMPROVE ABRADING EFFICIENCY”; U.S. Pat. Pub. No. 2013/0018398, entitled “ROTATIONAL ATHERECTOMY DEVICE WITH ELECTRIC MOTOR”; and U.S. Pat. No. 7,666,202, entitled “ORBITAL ATHERECTOMY DEVICE GUIDE WIRE DESIGN.” It is contemplated by this invention that the features of one or more of the embodiments of the present invention may be combined with one or more features of the embodiments of atherectomy devices described therein.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A method for measuring and providing real-time display of inner diameter, opposition force and compliance of a test biological conduit which may comprise a lesion, comprising:
providing at least one reference compliance curve generated by inflating a reference balloon with a set of known physical characteristics at a known and fixed inflation rate to a known and fixed volume and measuring the pressures associated with incremental volume changes;
measuring the outer diameter of the reference balloon during inflation and expansion;
mapping the outer diameters of the expanding reference balloon to the incremental volume changes and associated pressures;
providing at least one test compliance curve generated by inflating a test balloon, the test balloon having the set of known physical characteristics of the reference balloon, at the known and fixed inflation rate and fixed volume within the test biological conduit and measuring the pressure associated with incremental volume changes;
comparing the at least one reference compliance curve with the at least one test compliance curve;
determining at least one inner diameter of the test biological conduit;
determining at least one opposition force of the test biological conduit on the expanding test balloon; and
determining at least one compliance measurement of the test biological conduit and/or lesion.

2. The method of claim 1, further comprising displaying in real time the determined at least one inner diameter, at least one opposition force and at least one compliance measurement.

3. The method of claim 1, wherein determining the inner diameter of the test biological conduit further comprises locating a point of divergence between the at least one reference compliance curve and the test compliance curve.

4. The method of claim 3, further comprising comparing the volume and pressure data at the located point of divergence with the mapped outer diameters of the expanding reference balloon to the incremental volume changes and associated pressures to determine the inner diameter of the test biological conduit.

5. The method of claim 1, wherein determining the opposition force of the test biological conduit on the expanding test balloon comprises calculating for at least one given volume, the pressure difference between the at least one reference compliance curve and the test compliance curve.

6. The method of claim 5, further comprising calculating the total area between the at least one reference compliance curve and the test compliance curve.

7. The method of claim 1, wherein determining the compliance of the test biological conduit further comprises calculating a slope of the test compliance curve, calculating a slope of the at least one reference compliance curve and comparing the test and reference compliance curve slopes, wherein the slopes are calculated either at a set of given volumes wherein the calculated slopes represent the pressure changes at the given volumes or at a set of given pressures wherein the calculated slopes represent the volume changes at the given pressures.

8. The method of claim 1, wherein the compliance curves, results of the at least one inner diameter, opposition force and compliance determinations are automatically calculated by a computing device and displayed on a display device operatively connected with the computing device.

9. A method for real-time evaluation of the progress and efficacy of an occlusion-removal procedure on a test biological conduit having a lesion, comprising:
providing at least one reference compliance curve generated by inflating a reference balloon with a set of known physical characteristics at a known and fixed inflation rate to a known and fixed volume and measuring the pressures associated with incremental volume changes;
measuring the outer diameter of the reference balloon during inflation and expansion;
mapping the outer diameters of the expanding reference balloon to the incremental volume changes and associated pressures;
providing at least one test compliance curve generated by inflating a test balloon, the test balloon having the set of known physical characteristics of the reference balloon, at the known and fixed inflation rate and fixed volume within the test biological conduit and measuring the pressure associated with incremental volume changes;
comparing the at least one reference compliance curve with the at least one test compliance curve;
determining at least one inner diameter of the test biological conduit;
determining at least one opposition force of the test biological conduit on the expanding test balloon;
determining at least one compliance measurement of the test biological conduit and/or lesion; and
determining whether the procedure is complete based on the inner diameter, opposition force of the biological conduit and the compliance of the test biological conduit and/or lesion.

10. The method of claim 9, further comprising displaying in real time the determined at least one diameter, at least one opposition force and at least one compliance measurement.

11. A system for measuring a biological conduit's inner diameter, opposition force and compliance, wherein the biological conduit may comprise a lesion, comprising:
a syringe having a known and fixed volume of inflation fluid therein;
an inflatable balloon in fluid communication with the syringe and positioned within the biological conduit;
a means for providing a constant and known inflation rate of the known and fixed volume of inflation fluid from the syringe to the inflatable balloon; and
a pressure transducer in operative communication with the inflatable balloon and capable of determining the pressure within the balloon during inflation.

12. The system of claim 11, wherein the means for providing a constant and known inflation rate of the inflation fluid from the syringe to the test balloon comprises a linear motor.

13. The system of claim 11, further comprising a programmable computing device comprising a memory wherein executable instructions and a database of pre-generated reference compliance curves are stored, a processor in operative communication with the memory and wherein the executable instructions are run, a data input in operative communication with the memory and the processor, a display in operative communication with the data input and the processor, wherein the computing device is in operative communication with the means for providing a constant and known inflation rate of the inflation fluid from the syringe to the test balloon and with the pressure transducer.

14. A method for measuring and providing real-time display of metrics for a biological conduit and/or when present a lesion within the biological conduit, comprising:
providing at least one reference compliance curve generated by inflating a reference balloon with a set of known physical characteristics at a known and fixed inflation rate to a known and fixed volume and measuring the pressures associated with incremental volume changes;
measuring the outer diameter of the reference balloon during inflation and expansion;
mapping the outer diameters of the expanding reference balloon to the incremental volume changes and associated pressures;
providing at least one test compliance curve generated by inflating a test balloon, the test balloon having the set of known physical characteristics of the reference balloon, at the known and fixed inflation rate and fixed volume within the test biological conduit and measuring the pressure associated with incremental volume changes;
comparing the at least one reference compliance curve with the at least one test compliance curve;
determining at least one inner diameter of the test biological conduit by:
locating a point of divergence between the at least one reference compliance curve and the test compliance curve;
comparing the volume and pressure data at the located point of divergence with the mapped outer diameters of the expanding reference balloon to the incremental volume changes and associated pressures to determine the inner diameter of the test biological conduit; and
displaying the determined inner diameter of the test biological conduit in real time.

15. The method of claim 14, further comprising:
determining an opposition force of the test biological conduit on the expanding test balloon comprises calculating for at least one given volume, the pressure difference between the at least one reference compliance curve and the test compliance curve.

16. The method of claim 15, further comprising displaying the determined opposition force of the test biological conduit in real time.

17. The method of claim 16, further comprising calculating the total area between the at least one reference compliance curve and the test compliance curve to obtain total opposition force.

18. The method of claim 17, further comprising displaying the determined total opposition force of the test biological conduit in real time.

19. The method of claim 14, further comprising:
determining compliance of the test biological conduit and/or lesion by calculating a slope of the test compliance curve, calculating a slope of the at least one reference compliance curve and comparing the test and reference compliance curve slopes, wherein the slopes are calculated either at a set of given volumes wherein the calculated slopes represent the pressure changes at the given volumes or at a set of given pressures wherein the calculated slopes represent the volume changes at the given pressures.

20. The method of claim 19, further comprising displaying the determined compliance of the test biological conduit and/or lesion in real time.
